(19) United States
(12) Patent Application Publication
(10) Pub. No.: US 2019/0062402 A1
(43) Pub. Date: Feb. 28, 2019

(54) ACVR1-FC FUSION PROTEIN, PREPARATION METHOD THEREFOR, AND APPLICATION THEREOF

(71) Applicant: SHANGHAI KANDA BIOTECHNOLOGY, Shanghai (CN)

(72) Inventors: Yi CHEN, Shanghai (CN); Zeling CAI, Shanghai (CN); Keqin ZHANG, Shanghai (CN)

(21) Appl. No.: 15/755,752

(22) PCT Filed: Aug. 8, 2016
(86) PCT No.: PCT/CN2016/093910

§ 371 Date: Sep. 20, 2018

(30) Foreign Application Priority Data
Aug. 27, 2015 (CN) 201510534850.4

(51) Int. Cl. C07K 14/71; A61P 19/08

(52) U.S. Cl. CPC C07K 14/71; A61P 19/08; A61K 38/00; C07K 2319/02; C07K 2319/30

(57) ABSTRACT

The present invention provides an ACVR1-Fc fusion protein, a nucleic acid sequence encoding said fusion protein, a vector or a host cell comprising said encoding sequence, a method for producing said fusion protein, and use of any of the above in prevention and/or treatment of diseases or conditions associated with ACVR1 abnormality (e.g. ACVR1 mutation and/or over-activation).

### FIELD OF INVENTION

This application belongs generally to the fields of biotechnology and medicine.

Specifically, the application is directed to a fusion protein of activin A receptor type I and Fc (“ACVR1-Fc”), production of the fusion protein and its uses in prevention and/or treatment of diseases or conditions associated with ACVR1 abnormality (e.g. mutation and/or over-activation), such as pleonosteosis-associated diseases, diffuse endogenous cerebral bridge glioma, ovarian cancer, etc.

### BACKGROUND OF THE INVENTION

“ACVR1” refers to Activin receptor type IA (ActRIA), a subtype of bone morphogenetic protein I (BMPI) receptors, and is also known as Activin receptor-like kinase 2 (“ALK2”).

ACVR1 belongs to type-I class of the superfamily of transforming growth factor β (TGF-β) receptors. The receptor protein is composed of an extracellular domain, a transmembrane domain and an intracellular domain. The C terminal of the intracellular domain acts as a Serine/Threonine protein kinase to transmit signals downstream. The intracellular domain has a GS region close to the membrane. The extracellular domain transmits signals into the cell upon stimulation. There are two ACVR1 signaling pathways: one is via directly binding to a bone morphogenetic protein (e.g. BMP-2 or BMP-6); and, the other one, which is also the major one, involves a bone morphogenetic protein (e.g. BMP-4) binding to the type II member of the superfamily TGFβ receptors (ACVR2) and then the cytokine-bound ACVR2 binding to ACVR1, whereby transmitting the signal into the cell.

Fibrodysplasia ossificans progressive (FOP), also known as myositis ossificans progressive (MOP), is a disastrous and rare congenital disabling disease, which is characterized in progressive heterotopic ossification induced by spontaneous muscle inflammation or muscle injury, and which may lead to synarthrosis and impaired mobility^{[1]}.

There is no established or effective therapy for this disease yet. Excision of ectopic bone usually induces relapse of the lesions in situ or deterioration. In case of early diagnosis, the clinic treatment usually includes prevention of deterioration, modulation of local functionality, anti-inflammation, etc.^{[2]} It has been reported that glucocorticoids, non-steroidal anti-inflammatory drugs (NSAIDs), bisphosphonates, rosiglitazone and radiotherapy have certain effects in some patients, which however lacks significance.

The study on FOP had been slow until the recent booming of development. Particularly, the following are several new findings about the pathogenesis:

I) Part of the cells involved in the genesis of FOP has been identified.

Though there have been observed infiltration of several cells (including monocytes, macrophages, mastocytes and T/B lymphocytes) in sites of skeletal muscle inflammation, it remains vague which is(are) the critical cell(s) (i.e., the progenitors of chondrocytes and osteoblasts) that contribute(s) to the disease. Lounev et al. [3] reported that lineage tracing in transgenic mice exhibiting a phenotype close to FOP revealed that about 40-50% of the chondrocytes and osteoblasts in the lesion area have the marker protein Tie2, which is specific for vascular endothelial cells. Further, Medici et al.^{[4]} have shown that human umbilical vein endothelial cells (HUVECs) transfected with the R206H mutant can differentiate into chondrocytes and osteoblasts in vitro, which indicated that vascular endothelial cells are involved in FOP. Still, about 50% of the cells involved are yet to be identified.

II) Mutation in ACVR1 gene is pivotal to the genesis of FOP.

In 2006, studies showed that mutation in ACVR1, the subtype of bone morphogenetic protein I (BMPI), is directly associated with the genesis of FOP.

As seen in a group (more than 70 cases) in China, up to 98.4% of the patients have the heterozygous single-base mutation (617G>A) in the exon of ACVR1 gene, which results in the substation of the arginine at position 206 by histidine (R206H) and increased ACVR1 activity. ACVR1 is a single-transmembrane protein in structure, having the sequence as set forth in FIG. 1, wherein the R206H mutation resides in the glycine/serine-abundant region (GS region, residues 178-207). This region is highly conserved among quite a number of species (including human beings), and is thought to be functionally important.

Molecular modeling of ACVR1 protein demonstrated^{[1]} that the mutation (R206H) leading to increased ACVR1 activity resides in the GS region close to the intracellular domain. The arginine (R) at position 206 forms a small side chain closely aligning with the a helix backbone, which stabilizes the molecular structure. While in the FOP patients, this arginine is substituted by histidine (H), and the latter protrudes away from the a helix backbone and thus makes the molecule instable. This can be seen as demonstrated by the increased activity of P38MAPK signaling^{[5]} (without increase in BMP-Smad signaling) downstream to lymphocyte receptors in patients, and by the increased activity in both the BMP-Smad and the BMP-MAP signaling pathways in dental pulp cells in vitro^{[6]}. These suggest that the R206H mutation leads to a constitutively active ACVR1 in patients.

III) FOP models in animal are established.

It is difficult to build animal models with heterozygous mutation or knock-in mutation in ACVR1 gene close to the real conditions in patients. Still, three animal models have been established which are useful in study. There have been reported several animal models with FOP-like phenotypes, which include the followings:

(A) With the knowledge that the Q207D mutation in ALK2 transforms the protein to a constitutively active form, Fukuda et al.^{[7]} made transgenic animals with a Q207D mutant of ALK2, which ended up in premature death of the transgenic embryo in midtrimester. The failure indicated that models of systematic R206H mutation in mouse are unguaranteed. Yu et al.^{[8]} transfected mice already modified to conditionally express ALK2 Q207D with adenovirus containing Cre enzyme (Ad.Cre) via intramuscular injection to induce expression of ALK2 Q207D in skeletal muscles and myositis (adenovirus induces myositis), and thereby obtained part of the phenotypes in FOP patients (i.e., ossification in muscle, limited joint mobility).

(B) Glaser et al. ^{[9]} implanted into abdominal muscles of mice Matrigels comprising BMP4 or BMP2, which induced heterotopic ossification in the implant area, similar to the case in FOP patients.

(C) Kan et al^{[10]} reported that transgenic mice transformed with neuron-specific enolase (NSE) promoter-BMP4 over-express BMP4 in neuromuscular junction, which leads to skeletal muscle inflammation and heterotopic ossification (definitely accompanied by brain tissue abnormality)^{[11]}.

Though these animal models are less accurate in reflecting the abnormalities in FOP patients, they are useful in development of treatment for FOP. Particularly, the models in (A) are quite close to human FOP in pathogenesis and pathogenic pathway, wherein ligands to ACVR1 or ACVR2 play a role in triggering genesis of the disease and also in disease development.

Despite the studies in mechanism and therapy of FOP, there is still the need for development of effective medicaments and therapies.

Besides causing FOP, ACVR1 mutation is also known as associated with high-grade glioma (HGG, also known as “pediatric brain tumor”). In 2014, almost at the same time, four groups (including those in the United States and Europe) all found that in patients diagnosed with diffuse endogenous cerebral bridge glioma (DIPG, a subtype of high-grade glioma), 20-30% developed recurring ACVR1 gene mutations^{[12-15]}. Analysis of ACVR1 gene mutations in DIPG patients reveals that they are closely similar to the mutations in ACVR1 gene in FOP patients, which also lead to sustained activation of the BMP/TGFβ signaling pathway of ACVR1 protein. 15-20% of pediatric brain tumors and spinal cord tumors belong to high-grade glioma, which are currently treated by surgery, radiotherapy and chemotherapy, with a long-term survival less than 20%.

Besides, studies showed that normal ACVR1 gene and protein are also associated with tumors. It has been reported that^{[16-17]} ovarian cancer patients have a higher blood level of Stress Induced Phosphoprotein 1 (STIP1) than normal. STIP1 is secreted by ovarian cancer cells, via autocrine or paracrine. It binds to ACVR1 protein on the surface of ovarian cancer cells, activates the SMAD signaling pathway, and promotes the growth of ovarian cancer cells.

Generally, ACVR1 protein is a target not only for development of therapy for disastrous and rare diseases (e.g. FOP and DIPG) but also for development of therapy for cancers of high incidence (e.g. ovarian cancer). There is a need for medicaments and methods for prevention and/or treatment of diseases and/or conditions associated with ACVR1 abnormality (e.g. mutation and/or over-activation).

### SUMMARY OF THE INVENTION

The present disclosure provides a biologically active ACVR1-Fc fusion protein, production of the fusion protein and its uses in prevention and/or treatment of diseases or conditions (e.g. FOP, DIPG, ovarian cancer, etc.) associated with ACVR1 abnormality (e.g. mutation and/or over-activation).

In the first aspect, the present disclosure provides a fusion protein comprising the following elements:

(a) an ACVR1 element, having the amino acid sequence of ACVR1 or a functionally active fragment thereof;

(b) an Fc element, comprising a human IgG Fc fragment;

(c) optionally, a signal peptide element; and

(d) optionally, linker peptide sequence(s) between any two of the above.

In some embodiments, the fusion protein is composed of elements (a), (b) and (c).

In some embodiments, the ACVR1 element is capable of binding to BMP-2.

In some embodiments, the ACVR1 element comprises the sequence of the extracellular domain of ACVR1.

In some embodiments, the ACVR1 element is selected from the group consisting of:

(i) one having the sequence of SEQ ID NO: 4;

(ii) one having a sequence containing one or more deletions, substitutions and/or additions relative to SEQ ID NO: 4 and having the same biological activity as the sequence of SEQ ID NO: 4; and

(iii) one having a sequence more than 90% homologous to SEQ ID NO:4 and having the same biological activity as the sequence of SEQ ID NO: 4.

In some embodiments, the Fc element comprises an Fc fragment of human IgG γ1, IgG γ2, IgG γ3 or IgG γ4. The Fc element comprises the hinge region, the CH2 region and the CH3 region.

In some embodiments, the Fc element is selected from the group consisting of:

(i) one having the sequence of SEQ ID NO: 6;

(ii) one having a sequence containing one or more deletions, substitutions and/or additions relative to SEQ ID NO: 6 and having the same biological activity as the sequence of SEQ ID NO: 6; and

(iii) one having a sequence more than 90% homologous to the sequence of SEQ ID NO:6 and having the same biological activity as the sequence of SEQ ID NO: 6.

In some embodiments, the signal peptide element is selected from the group consisting of the signal peptide of CD33 protein (preferably one having the sequence of SEQ ID NO: 2) or any other surface antigen signal proteins, a signal peptide of an antibody protein or a signal peptide of a secretive protein.

In some embodiments, the linker peptide sequence usually has a length of 1 to 50 amino acids, such as 5 to 50, 5 to 40, 10 to 40 amino acids.

In some embodiments, the fusion protein comprises the elements arranged in an order, in the direction of 5′ terminus to 3′ terminus, selected from the followings, wherein (d), (d1) and (d2) independently represent identical or different linker peptide sequences:

(a)-(b); (b)-(a); (c)-(a)-(b); (c)-(b)-(a); (a)-(d)-(b); (b)-(d)-(a);

(c)-(d)-(a)-(b); (c)-(a)-(d)-(b); (c)-(d)-(b)-(a); (c)-(b)-(d)-(a);

(c)-(d1)-(a)-(d2)-(b); and (c)-(d1)-(b)-(d2)-(a).

In some embodiments, the fusion protein posseses one or more activities selected from the followings: binding to the same cytokine(s) as the native ACVR1 does, binding the complex of cytokine(s) and ACVR2, inhibiting phosphorylation of protein Smad-1/5/8, inhibiting phosphorylation and activation of p38 MAP kinase, inhibiting osteogenic differentiation, inhibiting chondrogenic differentiation, and reducing the calcium ion level in intercellular matrix.

In some embodiments, the elements of the fusion protein are each independently selected as in the followings:

an ACVR1 element having the sequence of SEQ ID NO: 4;

an Fc element having the sequence of SEQ ID NO:6; and/or

a signal peptide having the sequence of SEQ ID NO:2.

In a preferred embodiment, the DNA molecule has the nucleotide sequence as set forth in SEQ ID NO: 1.

In some embodiments, the fusion protein is selected from the group consisting of

(i) one having the sequence of SEQ ID NO: 8;

(ii) one having a sequence containing one or more deletions, substitutions and/or additions relative to SEQ ID NO: 8 and having the same biological activity as the sequence of SEQ ID NO: 8; and

(iii) one having a sequence more than 90% homologous to the sequence of SEQ ID NO:8 and having the same biological activity as the sequence of SEQ ID NO: 8.

In a second aspect, the disclosure provides an isolated nucleic acid molecule being coding sequence of the fusion protein, or the complementary sequence of the coding sequence.

In some embodiments, the nucleic acid molecule comprises the sequence of SEQ ID NO: 3, the sequence of SEQ ID NO: 5 and optionally, the sequence of SEQ ID NO: 1.

In some embodiments, the nucleic acid molecule is selected from the group consisting of

(i) one having the sequence of SEQ ID NO: 7;

(ii) one having a sequence containing one or more amino acid deletions, substitutions and/or additions relative to SEQ ID NO: 7 and having the same biological activity as the sequence of SEQ ID NO: 7; and

(iii) one having a sequence more than 90% homologous to the sequence of SEQ ID NO:7 and having the same biological activity as the sequence of SEQ ID NO: 7.

In a third aspect, the disclosure provides a vector, which comprises the nucleic acid molecule of the invention.

In some embodiments, the vector is selected from those that are capable of effectively expressing recombinant proteins in bacteria, fungi, yeasts, plant or mammalian cells.

In some embodiments, the vector comprises an expression-regulatory element operably linked to the nucleic acid molecule.

In a fourth aspect, the disclosure provides a host cell, which comprises the vector of the invention.

In some embodiments, the host cell is selected from the group consisting of CHO DG44, CHO-S, NS/0 cells and other suitable mammalian cells.

In a fifth aspect, the disclosure provides a method of producing the fusion protein of the invention, which comprises:

(a) culturing the host cell of the invention under a suitable condition such that the fusion protein is expressed, and

(b) harvesting the fusion protein.

In some embodiments, the method further comprises one or more of the following steps: introducing the nucleic acid molecule of the invention into a suitable vector to obtain a vector of the invention; introducing the vector into a suitable host cell to obtain a host cell of the invention; separating and/or purifying the fusion protein via protein A affinity chromatography, anion-exchange chromatography, cation-exchange chromatography and/or hydrophobic chromatography.

In a sixth aspect, the disclosure provides uses of the fusion protein, the nucleic acid molecule, the vector and/or the host cell in manufacturing a medicament for prevention and/or treatment of diseases or conditions associated with ACVR1 abnormality (e.g. mutation and/or over-activation).

In some embodiments, the diseases or conditions include pleonosteosis-associated diseases, and cancers associated with ACVR1 mutation and/or over-activation.

In some embodiments, the pleonosteosis is caused by over-activated ACVR1 and/or ACVR2 signaling pathway(s).

In some embodiments, the pleonosteosis-associated disease or condition is selected from the group consisting of fibrodysplasia ossificans progressiva, restrictive myositis ossificans (acquired myositis ossificans traumatica), cartilage hyperostosis, and hyperostosis.

In some embodiments, the cancer is selected from the group consisting of high-grade glioma, such as diffuse endogenous cerebral bridge glioma (also known as pediatric brain tumor), and ovarian cancer.

In a seventh aspect, the disclosure provides a pharmaceutical composition comprising: active ingredient(s) selected from the group consisting of a fusion protein of the invention, a nucleic acid molecule of the invention, a vector of the invention and/or a host cells of the invention; and a pharmaceutically acceptable vehicle.

In some embodiments, the pharmaceutical composition is used for prevention and/or treatment of diseases or conditions associated with ACVR1 abnormality (e.g. mutation and/or over-activation of ACVR1).

In some embodiments, the disease or condition is selected from the group consisting of pleonosteosis-associated diseases, and cancers associated with ACVR1 mutation and/or over-activation. In some embodiments, the pleonosteosis is caused by over-activated ACVR1 and/or ACVR2 signaling pathway(s). In some embodiments, the pleonosteosis-associated disease or condition is selected from the group consisting of fibrodysplasia ossificans progressiva, restrictive myositis ossificans (acquired myositis ossificans traumatica), cartilage hyperostosis and hyperostosis.

In some embodiments, the cancer is selected from the group consisting of high-grade glioma, such as diffuse endogenous cerebral bridge glioma (also known as pediatric brain tumor), and ovarian cancer.

In some other aspects, the disclosure further provides a method of preventing and/or treating diseases or conditions associated with ACVR1 abnormality (e.g. mutation and/or over-activation), wherein the method comprises administering to a subject in need of the treatment a therapeutically effective amount of a fusion protein, a nucleic acid molecule, a vector and/or a host cell of the invention.

In some embodiments, the disease or condition is selected from the group consisting of pleonosteosis-associated diseases, and cancers associated with ACVR1 mutation and/or over-activation. In some embodiments, the pleonosteosis is caused by over-activated ACVR1 and/or ACVR2 signaling pathway(s). In some embodiments, the pleonosteosis-associated disease or condition is selected from the group consisting of fibrodysplasia ossificans progressiva, restrictive myositis ossificans (acquired myositis ossificans traumatica), cartilage hyperostosis, and hyperostosis.

In some embodiments, the cancer is selected from the group consisting of high-grade glioma, such as diffuse endogenous cerebral bridge glioma (also known as pediatric brain tumor), and ovarian cancer.

In some embodiments, the method further comprises combined use of an additional medicament or therapy for prevention and/or treatment of diseases or conditions associated with ACVR1 abnormality (e.g. mutation and/or over-activation).

In some embodiments, the method is used to prevent and/or treat FOP. The method may further include applying simultaneously or sequentially an additional treatment of FOP, wherein said additional treatment may include, for example, prevention of secondary damages, modulation of local functionality, anti-inflammation, administration of glucocorticoids, non-steroidal anti-inflammatory drugs NSAID, bisphosphonates and/or rosiglitazone, and radiotherapy.

In some embodiments, the method is used to prevent and/or treat cancers, which may further include applying simultaneously or sequentially an additional treatment of cancer, like radiotherapy, chemotherapy, surgery, etc.

The technical solutions and the features in one or several of the above embodiments can be recombined and/or reorganized without departing from the spirit and scope of the invention as claimed. Additional aspects and advantages of the present invention would be obvious in view of the present disclosure.

### BRIEF DESCRIPTION OF THE DRAWINGS

In the following, the inventions are specified with reference to the drawings. It should be understood that the drawings are provided only for description, with no intention to limit the scope of the invention.

FIG. 1: Structure of protein ACVR1. As depicted, the segment of amino acids (aa) 1-20 corresponds to the transmembrane signal peptide; aa 21-123 corresponds to the extracellular domain (yellow); aa 124-146 corresponds to the transmembrane sequence (boxed); aa 147-509 corresponds to the intracellular domain, wherein aa 178-207 corresponds to the glycine/serine-abundant region (Glycine/Serine, GS region) (green), aa 208-502 corresponds to the Serine/Threonine protein kinase region.

FIG. 2: Construction of the recombinant ACVR1-Fc fusion protein. FIG. 3: The nucleic acid sequence and the amino acid sequence of ACVR1-Fc. Therein, the segment of aa 1-16 corresponds to the signal peptide of human CD33, aa 17-119 correspond to the extracellular domain of human ACVR1; aa 120-351 corresponds to the Fc fragment consisting of aa 236-437 of human IgG γ1 chain.

FIG. 4: Construction of the recombinant adenovirus vector, wherein:

a: construction of the adenovirus plasmid expressing ACVR1 R206H;

b: an optical-microscopic picture of normally cultured HUVEC cells;

c: a fluorescent-microscopic picture of HUVECs infected with recombinant adenovirus expressing ACVR1 R206H;

FIG. 5: SDS-PAGE electrophoresis of the ACVR1-Fc fusion protein purified via protein A affinity chromatography. Three μg protein was loaded onto a 4-12% NuPAGE SDS-PAGE electrophoresis, and the gel was then stained with coomassie brilliant blue R-250. In the order from left to right, Lane 1 corresponds to the non-reductive electrophoresis, Lane 2 the reductive electrophoresis and Lane 3 the MW marker.

FIG. 6: HPLC-SEC of the ACVR1-Fc fusion protein purified via protein A affinity chromatography, wherein the red line corresponds to the ACVR1-Fc fusion protein, the green line corresponds to the fusion protein of the extracellular domain TNFR2 and Fc (CELGEN BIOPHARMA, Shanghai, China), which serves as the control, and the blue line corresponds to the MW marker for gel filtration.

FIG. 7a: the specific binding of ACVR1-Fc fusion protein to BMP-2 studied by ELISA.

FIG. 7b: the specific binding of ACVR1-Fc fusion protein to different proteins in the BMP/TGFβ signaling pathway studied by ELISA.

FIG. 8: Modeling of osteogenic differentiation in HUVECs:

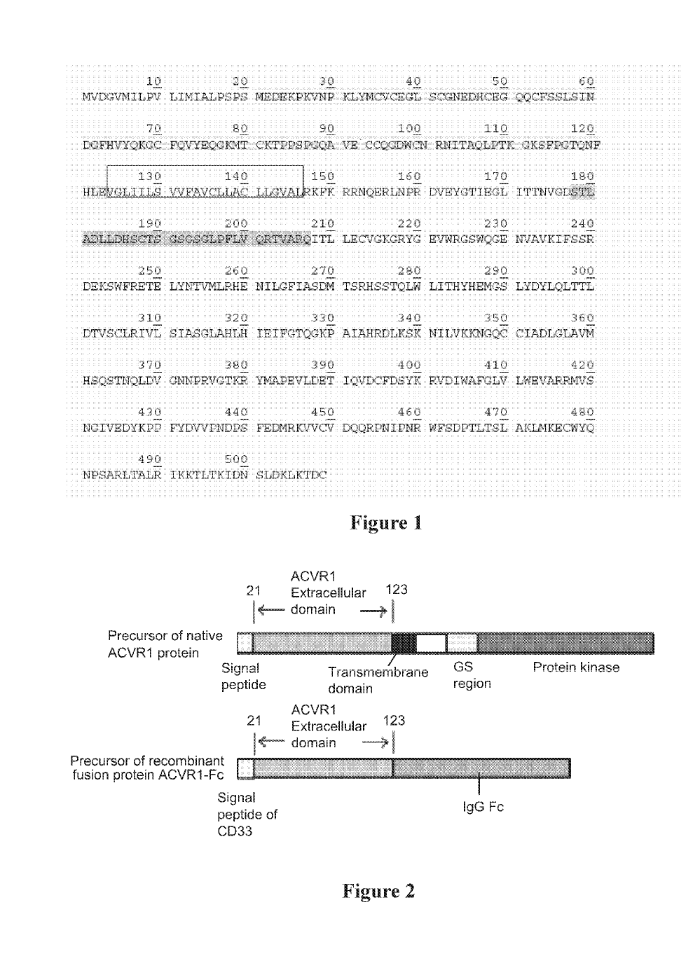

a: HUVECs were infected with ACVR1 R206H-adenovirus; 5 days later, the cells were transferred into the osteogenesis inducing medium and incubated for additional 7 days, and then stained with ALP;

b: HUVECs infected with ACVR1 R206H-adenovirus were incubated in the osteogenesis inducing medium for 21 days, and then stained with Alizarin Red;

c: HUVECs were infected with ACVR1 R206H-adenovirus; 5 days later, the cells were transferred into the osteogenesis inducing medium and incubated for additional 14 days, and then stained with Alcian Blue. The cells were examined by taking pictures under a bright field microscope.

FIG. 9: study of ACVR1-Fc's activity on inhibiting osteogenic differentiation in HUVECs by ALP staining. On day 7 of differentiation culturing, the HUVECs were stained with ALP to detect osteogenic differentiation. Recombinant human immunoglobulin Fc (Chimerigen Laboratories, Cat.# CHI-HF-210 IgG1) was used as the control:

a: differentiation medium containing 3 μg/ml control (recombinant human IgG 1 Fc);

b: differentiation medium containing 1.5 μg/ml ACVR1-Fc fusion protein;

c: differentiation medium containing 3 μg/ml ACVR1-Fc fusion protein.

FIG. 10: study of ACVR1-Fc activity on inhibiting osteogenic differentiation in HUVECs by Alizarin Red staining. On day 21 of differentiation, the HUVECs were stained with Alizarin Red to detect osteogenic differentiation:

a: differentiation medium containing 3 μg/ml Fc protein as control (recombinant human IgG1Fc);

b: differentiation medium containing 1.5 μg/ml ACVR1-Fc fusion protein;

c: differentiation medium containing 3 μg/ml ACVR1-Fc fusion protein.

FIG. 11: study of ACVR1-Fc's inhibitory activity on chondrogenic differentiation in HUVECs by Alcian Blue staining. On day 21 of differentiation, the HUVECs were stained with Alcian Blue to detect chondrogenic differentiation:

a: differentiation medium containing 3 μg/ml Fc protein as control (recombinant human IgG1Fc);

b: differentiation medium containing 1.5 μg/ml ACVR1-Fc fusion protein;

c: differentiation medium containing 3 μg/ml ACVR1-Fc fusion protein.

FIG. 12: ACVR1-Fc's inhibitory activity on osteogenic differentiation by Atomic absorption spectrometry assay. Cells were induced to differentiate in the medium supplemented with Fc protein as the control (recombinant human IgG1Fc) or the ACVR1-Fc fusion protein; 21 days later, the cells were harvested and subjected to the Calcium ion level detection via atomic emission spectrometer. ***: P<0.001.

FIG. 13: ACVR1-Fc's effects on expression of the marker proteins of osteogenic differentiation and chondrogenic differentiation by Western blotting:

a: Western blotting showing ACVR1-Fc's effects on the expression of five osteogenesis markers;

b: Inhibition of each of the markers was calculated, taking the expression of GAPDH as the baseline.

FIG. 14: ACVR1-Fc's inhibitory activity on phosphorylation of protein Smad-1/5/8 and p38 MAP kinase:

a: Western blotting of ACVR1-Fc's effects on inhibiting the phosphorylation of Smad-1/5/8 and p38MAP;

b: quantitative analysis of the inhibition of phosphorylation of Smad-1/5/8 protein by ACVR1-Fc via Western blotting;

b and c: quantitative analysis of the inhibition of phosphorylation of p38MAP protein by ACVR1-Fc via Western blotting;

***: P<0.001.

### DETAILED DESCRIPTION OF THE INVENTION

The inventors, through extensive and intensive studies, constructed an expression vector of ACVR1-Fc fusion protein, obtained an ACVR1-Fc fusion protein and characterized the superior biological activities of the fusion protein, which promises to be a new approach for preventing and treating diseases or conditions associated with ACVR1 abnormality (e.g. ACVR1 mutation and/or over-activation). For instance, the fusion protein of the invention can effectively inhibit the activation of the ACVR1 and the ACVR2 pathways, and thereby inhibit osteogenic differentiation and chondrogenic differentiation, which makes it useful in prevention and/or treatment of pleonosteosis-associated diseases and/or conditions (e.g. fibrodysplasia ossificans progressiva FOP) caused by over-activated ACVR1 and/or ACVR2 signaling pathway(s).

In the present disclosure, each reference to a range equals to specific disclosure of each of the values in between and also every subsets in between. All the features and elements, though being specified in context an embodiment or example for purpose of explanation and exemplification, can be recombined and reorganized without departing the spirit and scope of invention. In the present disclosure, the features and elements, beyond the specified specific examples, extend to include equivalents in form and in identity, which can be used in an exchangeable way. Unless otherwise indicated, the disclosed features are exemplary examples for the equivalents or similar features.

As used herein, the terms “comprises/comprise/comprising”, “has/have/having” and “includes/include/including”, including their grammar cognates, are used in an exchangeable way and each include the meaning of or equal to “comprise”, “essentially consist of . . . ”, “substantially consist of . . . ” and “consist of . . . ”

As used herein, the term “isolated”, with reference to nucleic acid molecule and protein, means that the referenced material is separated from and thus substantially free of the substances (e.g. cell components) that are co-present in its naturally occurring environment. The isolated material is preferably homogeneous, in a dry or aqueous state. Purity and homogeneity can be determined via assays like polyacrylamide gel electrophoresis and high performance liquid chromatography. The terms “protein”, “peptide” and “polypeptide” are used in an exchangeable way and refer to a chain of two or more amino acids linked by one or more peptide bond(s) or amido bond(s), which may be optionally modified by, for instance, glycosylation and/or phosphorylation.

Fusion Protein and Elements Thereof

As used herein, unless otherwise specified, the term “fusion protein” refers to an isolated protein, which may be recombinantly produced by a host cell or be extracted in a purified form.

A fusion protein of the invention may comprise an element (a) and an element (b), optionally, an element (c), and ever further, optionally an element (d), as defined in the followings:

(a) an ACVR1 element, comprising the amino acid sequence of ACVR1 or a functionally active fragment thereof;

(b) an Fc element, comprising an Fc fragment of human IgG;

(c) optionally, a signal peptide element; and

(d) optionally, linker peptide(s) between any two of the above.

As used herein, the term “element” refers to an amino acid sequence incorporated as an integral part of the fusion protein.

In the present disclosure, the ACVR1 element (a) has an amino acid sequence substantially identical to the full-length sequence of the native ACVR1 or a variant thereof, or to the extracellular domain, and has substantially the same biological activity of the native ACVR1. The element (a) preferably has the sequence of the extracellular domain of ACVR1, and more preferably has the sequence of SEQ ID NO: 4.

In some embodiments, the ACVR1 element is selected from the group consisting of:

(i) one having the sequence of SEQ ID NO: 4;

(ii) one having a sequence containing one or more amino acid deletions, substitutions and/or additions relative to the sequence of SEQ ID NO: 4 and having the same biological activity as the sequence of SEQ ID NO: 4; and

(iii) one having a sequence more than 90% homologous to the sequence of SEQ ID NO:4 and having the same biological activity as the sequence of SEQ ID NO: 4.

As used herein, the term “Fc region” or “Fc fragment” refers to a fragment consisting of hinge region +CH2 region +CH3 region. In the present disclosure, the Fc element (b) has an amino acid sequence substantially the same as that of a native IgG Fc fragment or a variant thereof, and has substantially the same biological activity as the native Fc fragment. Besides the CH2 and the CH3 regions of IgG, the Fc element may further comprise the hinge region. The element (b) may be the Fc region from IgGγ1-4, preferably the Fc region from IgG γ1, more preferably one having the sequence of SEQ ID NO: 6.

In some embodiments, the Fc element is selected from the group consisting of:

(i) one having the sequence of SEQ ID NO: 6;

(ii) one having a sequence containing one or more amino acid deletions, substitutions and/or additions relative to SEQ ID NO: 6 and have the same biological activity as the sequence of SEQ ID NO: 6; and

(iii) one having a sequence more than 90% homologous to the sequence of SEQ ID NO:6 and having the same biological activity as the sequence of SEQ ID NO: 6.

In the present disclosure, “signal peptide element” (c) refers to an amino acid sequence that directs secretion, location and/or transportation of the fusion protein, which is usually 5-30 amino acids in length.

In some embodiments, the signal peptide element is the signal peptide of protein CD33 (preferably having the sequence of SEQ ID NO: 2), or any other signal peptide capable of guiding a protein's secretion into extracellular environment.

In the present disclosure, the “linker peptide sequence” (d) refers to a short peptide acting as a linkage between any two of the elements in the fusion protein, which is usually 1 to 50 (e.g. 5 to 50, 5 to 40, or 10 to 40) amino acids long. A person of ordinary skills in the art knows how to design a suitable linker peptide using conventional means and tools (e.g. PNAS 1998; 95: 5929-5934; Protein Eng, 2000; 13(5): 309-312; Protein Eng, 2003; 15(11): 871 - 879). Usually, the linker peptide does not or does not substantively interfere the correct folding and spatial confirmation of the fusion protein of the invention.

In the present disclosure, the fusion protein may comprise the elements arranged in any one of the following orders in the direction of 5′ to 3′:

(a)-(b); (b)-(a); (c)-(a)-(b); (c)-(b)-(a); (a)-(d)-(b); (b)-(d)-(a); (c)-(d)-(a)-(b);

(c)-(a)-(d)-(b); (c)-(d)-(b)-(a); (c)-(b)-(d)-(a); (c)-(d1)-(a)-(d2)-(b); and

(c)-(d1)-(b)-(d2)-(a),

Wherein, (a) represents the ACVR1 element; (b) represents the Fc element; (c) represents the signal peptide element; (d) represents a linker peptide sequence; (d), (d1) and (d2) independently represent linker peptide sequences that are identidal or different.

In the present disclosure, preferably, the fusion protein

(i) having the sequence of SEQ ID NO: 8;

(ii) having a sequence containing one or more amino acid deletions, substitutions and/or additions relative to SEQ ID NO: 8 and having the same biological activity as the sequence of SEQ ID NO: 8; or

(iii) having a sequence more than 90% homologous to the sequence of SEQ ID NO:8 and having the same biological activity as the sequence of SEQ ID NO: 8.

In some embodiments, the fusion protein posseses one or more of the following activities: binding to the same cytokine(s) as the native ACVR1 does, binding to the complex of cytokine(s) and ACVR2, inhibiting phosphorylation of protein Smad-1/5/8, inhibiting phosphorylation and activation of p38 MAP kinase, inhibiting osteogenic differentiation, inhibiting chondrogenic differentiation, reducing calcium ion level in intercellular matrix.

In the present disclosure, for each of said elements, also contemplated and included are variants of a protein, polypeptide or peptide designated as any one of the elements, which have the same or equivalent biological activity as the prototype protein, polypeptide or peptide. Variation includes but is not limited to one or more (usually 1-50, preferably 1-30, more preferably 1-20, most preferably 1-10) amino acid deletions, additions and/or substitutions compared to native amino acid sequence. The deletion(s) or addition(s) (insertion(s)) may occur in the C-terminal region and/or the N-terminal region, and is usually no more than 20 amino acids in number, preferably no more than 10, and more preferably no more than 5. As understood, substitution(s) can be made using amino acids of similary properties to retain activities of the prototype protein. Conservative amino acid substitutions are well-known, for example, as within each of the following five groups: aliphatic amino acids: glycine(G), alanine (A), valine (V), leucine (L), isoleucine (I); aromatic amino acids: phenylalanine (F), tyrosine (Y), tryptophan (W); sulfur-containing amino acids: methionine (M), cysteine (C); basic amino acids: arginine(R), Lysine (K), histidine(H); and acidic amino acids: aspartic acid (D), glutamic acid (E), asparagine (N), glutamine (Q). In addition, also contemplated are the fragments or derivatives of inhibitory factors and human albumin, which preferably retain the desired biological activities.

Said variants also include analogs of the proteins or polypeptides. Compared to the native protein, the analogs may distinguish in amino acid sequence and/or modification onto the primary sequence. These polypeptides include naturally occurring variants and artificially induced genetic variants. Induced variants can be obtained in various ways, such as radiation or exposure to a mutagenic agent to induce random mutagenesis, site-directed mutagenesis, and other mutagenesis techniques known in molecular biology. Analogs also include those containing a residue that is not a naturally occurring L-amino acid (e.g. a D-amino acid), and those containing a non-natually occuring or artificial amino acid (e.g. β or γ-amino cids). It should be understood that the above specification is exemplary, and has no effect of limiting the scope of polypeptides of the invention. Modification, not in sense of changing primary amino acid sequence, may include in vivo or in vitro chemical derivation of a polypeptide, such as acetylation and carboxylation. Modification also includes glycosylation, including those occuring in synthesis, processing or subsequent processing of a polypeptide, so as to obtain a glycosylated polypeptide. This can be conducted by exposing a polypeptide to an enzyme involved in glycosylation (e.g. a mammalian glycosylase or a deglycosylase). Modified forms include sequences that comprise one or more phosphorylated amino acid residue(s) (e.g. phosphotyrosine, phosphoserine, phosphothreonine).

Each of the elements of the invention, when being designated with a specified peptide or polypeptide, also includes the polypeptides substantially identical (homologous) to the specified one, such as a polypeptide that has an identity (homology) of at least 60%, 70%, 80%, 90%, 95%, 97%, 98%, 99% or above to the specified one.

Based on the amino acid sequences provided herein, a person of ordinary skills in the art would know how to obtain a fusion protein of the invention using conventional means and tools, such as recombinant techniques, artificial synthesis, etc. [e.g. Murray K M, Dahl SLAnn; Pharmacother 1997 November; 31(11):1335-8]. For instance, a fusion protein of the invention may be directly obtained via solid-phase synthesis, or via assembly of synthetic fragments into the full-length molecule.

Fusion Protein Coding Sequences, Vectors and Host Cells

The present disclosure provides an isolated nucleic acid molecule having a nucleic acid sequence encoding the fusion protein, or the complementary sequence thereof. The nucleic acid molecule encoding the fusion protein of invention can be fully synthesized or be assembled from fragments each encoding one or more elements of the protein.

The nucleic acid sequence of the invention can be obtained using any of the conventional means, including PCR amplification, recombinant techniques and artificial synthesis. In the case of PCR amplification, the primers can be designed according to the relevant nucleotide sequence(s) in this disclosure, especially the ORFs, and the templates can be obtained from commercially accessible or self-developed cDNA banks, which then give the target sequence via amplification. In case of a long target sequence, usually overlapping amplification, like two or more PCR processes, are conducted to give batches of different fragments which are then assembled into the full-length target. The sequence thus obtained can then be reproduced in bulk using, for example, recombinant techniques.

In the present disclosure, the nucleic acid molecule encoding the fusion protein may comprise the sequence of SEQ ID NO: 3, which encodes the ACVR1 element; the sequence of SEQ ID NO: 5, which encodes the Fc element; and optionally, the sequence of SEQ ID NO: 1, which encodes the signal peptide element.

In a preferred embodiment of the invention, the nucleic acid molecule has a sequence selected from the followings:

(i) one having the sequence of SEQ ID NO: 7;

(ii) one having a sequence containing one or more amino acid deletions, substitutions and/or additions relative to SEQ ID NO: 7 and having the same biological activity as the sequence of SEQ ID NO: 7;

(iii) one having a sequence more than 90% homologous to the sequence of SEQ ID NO:7 and having the same biological activity as the sequence of SEQ ID NO: 7; and

(iv) one hybridizing to any one of the above sequences under a stringent condition and being correspondingly functionally active.

The invention also includes nucleic acid sequences that are identical (homologous) or substantially identical (homologous) to a specified one, such as those having an identity (homology) of at least 60%, 70%, 80%, 90%, 95%, 97%, 98%, 99% or even higher. When two nucleic acid sequences are described as substantially identical/homologous to each other, they hybridize under a high stringent condition. Accordingly, the nucleic acid sequences of invention include those hybridizing to a specified one, especially for example the sequence of SEQ ID NO: 7, under a moderate stringent condition, and more preferably under a high stringent condition.

As used herein, the term the term “stringent condition” refers to:

(1) hybridization and elution at a low ionic strength and a high temperature, such as 0.2×SSC, 0.1% SDS, 60 ° C.; or

(2) hybridization in the presence of a denaturing agent (e.g. 50%(v/v) formamide), 0.1% calf serum/0.1% Ficoll, 42° C. etc.; or

(3) hybridization only occurring between sequences that are at least 50%, preferably at least 55%, 60%, 65%, 70%, 75%, 80%, 85%, 90% or more, and more preferably at least 95% homologous to each other.

The DNA sequence of the fusion protein of invention may be incorporated in a suitable expression vector, which is then transferred into a suitable host cell. The transformed host cells are cultivated in a culture, from which the fusion protein of invention can be separated and purified.

As used herein, the term “vector” includes plasmids, cosmids, expression vectors, clonal vectors, viruse vectors, etc. Examples include but are not limited to vectors capable of expression in eukaryotes (e.g. CHO cells, COS cells, etc.), vectors capable of expression in yeasts (e.g. Saccharomyces cerevisiae and Pichia pastoris), vectors capable of expression in insect cells (e.g. silk worms), and vectors capable of expression in prokaryotes. Commercially available vectors are useful here. For example, an expression vector according to the disclosure may be constructed using a commercially available vector by incorporating the nucleotide sequence encoding the fusion protein as operably linked to a regulatory sequence.

As used herein, the term “operably linked” refers to the association of DNA sequences in a single chain so that the function of one is affected by the other. In one instance, a DNA sequence of signal peptide (i.e, a leader sequence) is operably linked to a polypeptide-coding sequence, when the DNA sequence of signal peptide is expressed as part of the precursor to mediate secretion of the polypeptide. In another instance, a promoter is operably linked to a coding sequence, when it regulates transcription of the coding sequence. In a further instance, a ribosome bind site is operably linked to a coding sequence, if it is located so that the coding sequence is effectively expressed. Usually, “operably linked” means being adjacent and, in the case of a leader sequence, it means being adjacent within ORF.

As used herein, the term “host cell” includes prokaryotes and eukaryotes. Prokaryotic examples include Escherichia coli, Bacillus Subtilis, etc. Eukaryotic examples include yeasts, insect cells and mammalian cells. Preferably, the host cell is a eukaryote, like CHO DG44.

As used herein, the term “transform” refers to introduction of a nucleic acid molecule of interests into a host cell using whatever a suitable means as known in the art. Examples of transformation techniques, each maybe differentially prefer certain type(s) of host, include inter alia electroporation, transfection using chemicals like calcium chloride and DEAE-glucan, particle bombardment, lipofection, and infection (see, Sambrook et al., Molecular Cloning, 2n^{d} Version, 1989). In an embodiment, electroporation is preferred.

The host cells are cultivated under a suitable condition such that the fusion protein is expressed. The condition of cultivation, such as medium, temperature and time, can be determined via routine experimentation. The expression of said fusion protein can be detected by any suitable means and tools as known in the field, such as SDS-PAGE, Western blotting, etc. Finally, conventional protein separation and purification techniques may be used to give the purified fusion protein, such as centrifugation, precipitation, filtration, chromatography, etc. Specifically, useful chromatography techniques include affinity assay, gel filtration, ion exchange, hydrophobic chromatography, and reverse chromatography, etc. Isolation and purification of the fusion protein may also include combination between or among any of the above techniques.

Applications

The fusion protein, the coding sequence, the vector and the host cell comprising the coding sequence can be used as a medicament for prevention and/or treatment of a disease or a condition associated with ACVR1 abnormality (e.g. mutation and/or over-activation), such as a pleonosteosis-associated disease or condition, and a cancer associated with ACVR1 mutation and/or over-activation, etc.

Preferably, the pleonosteosis is caused by over-activated ACVR1 and/or ACVR2 signaling pathway(s). In some embodiments, the pleonosteosis-associated disease or condition is selected from the group consisting of fibrodysplasia ossificans progressiva, cartilage hyperostosis, hyperostosis, etc. Preferably, the cancer associated with ACVR1 mutation and/or over-activation is selected from the group consisting of high-grade glioma, such as diffuse endogenous cerebral bridge glioma, ovarian cancer, etc.

Accordingly, the disclosure also provides a pharmaceutical composition that comprises (a) an effective amount of the fusion protein of the invention, the coding sequence, the vector and/or the host cell comprising said coding sequence of the invention, and (b) a pharmaceutically acceptable vehicle.

As used herein, the term “an effective amount” or “an effective dosage” refers to an amount, which confers an effect or action on the treated subject (human or animal) and which can be tolerated by the subject. The term “pharmacologically acceptable” means that when a molecule or a composition is appropriately administrated to a subject (human or animal), it does not cause undesired effects, such as toxicity, irritation and allergia, and has a reasonable efficacy/risk ratio.

“A pharmaceutically acceptable vehicle” should be compatible with the fusion protein of invention. That is, the vehicle, when being mixed with the protein in a pharmaceutical composition, does not cause a substance reduction in pharmacological efficacy. Examples of pharmaceutically acceptable vehicles or components thereof include those as taught in Remington: The Science and Practice of Pharmacy 21^{st} Edition, Univ. of Sciences in Philadelphia (USIP), Lippincott Williams & Wilkins, Philadelphia, Pa., 2005.

The pharmaceutical composition may be formulated in various dosage forms. A beneficial dosage can be determined by a physician according to known factors, including race, age, body weight, specific condition or state of the disease to be treated and path of administration. Paths of administration include inter alia oral, intranasal, respiratory airway pathways.

In the instance that the therapeutic agent is a polynucleotide, it can be delivered in the form of a naked polynucleotide, a combination or conjugate with a drug delivery agent, and/or a recombinant plasmid or viral vector comprising same or a component comprised in the plasmid or viral vector. Examples of agents useful for drug delivery include inter alia Mirus M Transit TKO lipothrophic reagents, lipofectin reagents, cellfectins, cationic polymers (e.g. polylysine) and liposomes.

For an enhanced effect, the fusion protein of invention may be used in combination with an additional medicament or therapy. For instance, prevention and/or treatment of FOP using a fusion protein of the invention may also include simultaneous or sequential administration or application of an additional medicament or therapy of FOP. The additional medicament or therapy may include but not be limited to prevention of a secondary damage, modulation of local functionality, anti-inflammation, administration of glucocorticoids, non-steroidal anti-inflammatory drugs NSAID, bisphosphonates, and/or rosiglitazone and radiotherapy. For instance, prevention and/or treatment of cancers associated with ACVR1 mutation and/or over-activation using a fusion protein of the invention may also include simultaneous or sequential administration or application of an additional medicament or therapy of FOP, wherein the additional medicament or therapy may include but not be limited to radiotherapy, chemotherapy, surgery, etc.

Advantages

The present disclosure provides solutions to problems such as difficulty in obtaining effective expression of a recombinant protein, lack of high-expression stains, incorrect configuration and/or conformation leading to insolubility or aggregation or loss in biological activity. The present invention, in one aspect, provides a recombinant protein molecule comprising extracellular domain of a receptor, which allows for effective expression in mammalian cells.

The fusion proteins of invention have the advantages including stable expression, high yield, easiness in purification and highly biologically active, and can be advantageously used to prevent and/or treat diseases and/or conditions associated with ACVR1 abnormality (e.g. mutation and/or over-activation). For instance, the fusion proteins of invention can effectively inhibit osteogenic differentiation and chondrogenic differentiation, and are thus useful in prevention and treatment of pleonosteosis-associated diseases or condition. In addition, the fusion proteins of the invention are also capable of inhibiting genesis, development and metastasis of tumors (e.g. ovarian cancer) associated with ACVR1 mutation and/or over-activation.

### EXAMPLES

The invention would be further illustrated by referring to the following examples. It should be understood that the examples are provided only for the purpose of illustration, rather than limiting scope of the invention. Various forms of modification and variation would be obvious to the skilled in the art, which are all included in scope of the invention.

In the following Examples, unless otherwise specified, conventional conditions and methods are used, for example, as taught in Molecular Cloning—A Laboratory Manual (3^{rd} version, Cold Spring Harbor Laboratory Press, New York, 1989) or as taught in the manufacturer's instruction. DNA sequencing can be performed using conventional means or by a commercial institute.

Unless otherwise specified, all the percentages and parts are calculated on a weight basis. Unless otherwise defined, technical and scientific terms used herein have the same meaning as commonly understood by a person of ordinary skill in the art. Particularly, the materials and methods as specified are exemplary, and various equivalents can be used.

### Example 1

### Construction of Expression Plasmid of the Fusion Protein

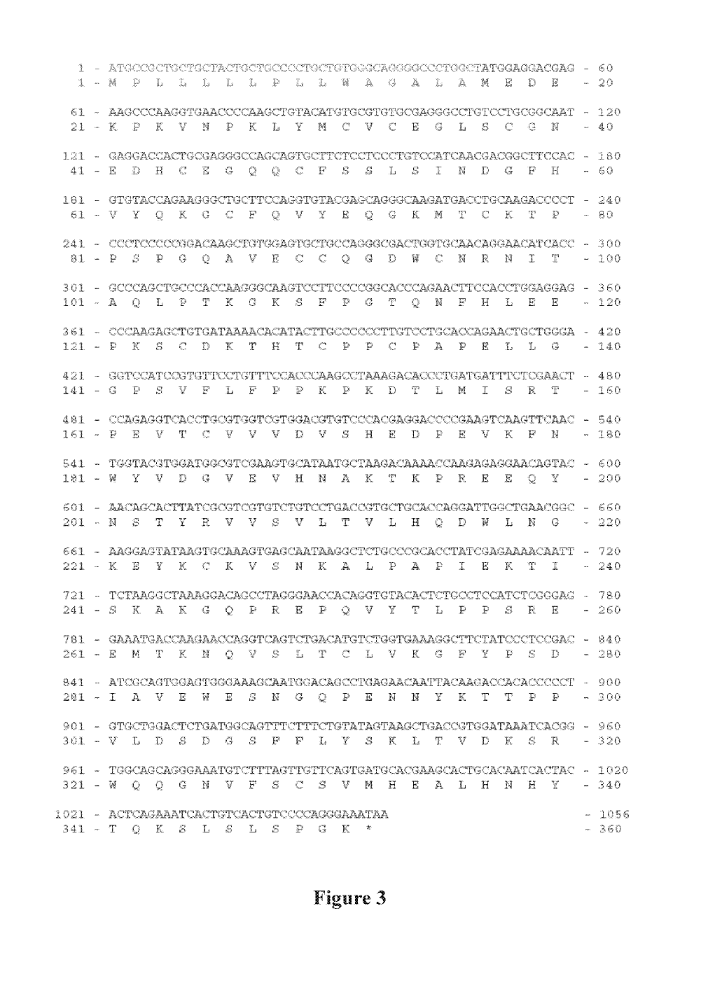

The ACVR1-Fc expression gene consists of three fragments (FIG. 2 and FIG. 3), which are, as ordered from the 5′ terminus to the 3′ terminus:

Fragment 1: the signal peptide sequence of protein CD33 at the 5′ terminus (the coding sequence is as set forth by SEQ ID NO: 1 and the corresponding amino acid sequence is as set forth by SEQ ID NO: 2);

Fragment 2: expression gene of the ACVR1 extracellular domain (amino acids 21-123) in the middle (the coding sequence is as set forth by SEQ ID NO: 3 and the corresponding amino acid sequence is as set forth by SEQ ID NO: 4); and

Fragment 3: the sequence encoding the amino acid sequence of human IgG γ1 at the 3′ terminus (the coding sequence is as set forth by SEQ ID NO: 5 and the corresponding amino acid sequence is as set forth by SEQ ID NO: 6), which encodes the fragment spanning amino acid residues 216 to 447 of human IgG γ1, including the hinge region, the CH2 region and the CH3 region (i.e., hinge+CH2+CH3).

The three genes were individually produced by polymerase chain reaction (PCR), and then assembled via overlap extension PCR. The polymerase chain reaction was conducted using high-fidelity polymerase Plantium pfx (Invitrogen). The PCR condition was designed by following the manufacturer's instruction and adapting to the selected type of reaction. The PCR fragments were purified using the gel DNA fragment kit (Qiagen).

Synthesis of Fragment 1

The template for PCR amplification of Fragment 1 comprised the nucleotide sequence (SEQ ID NO: 1) coding for the 16-amino acid signal peptide of CD33 protein.

The 5′-primer CMV-P comes from the plasmid vector, which has the sequence of

(SEQ ID NO: 9)
5′-CGCAAATGGGCGGTAGGCGTG-3′;

and

The 3′-primer SP-3 has the sequence of 5′-AGCCAGGGCCCCTGCC-3′ (SEQ ID NO: 10).

Synthesis of the cDNA of Fragment 2

The template plasmid for PCR amplification of Fragment 2 comprised the full-length gene of ACVR1′s extracellular domain (SEQ ID NO: 3).

The 5′-primer ACVR1-5 has the sequence of

(SEQ ID NO: 11)
5′-GGGCAGGGGCCCTGGCTATGGAGGACGAGAA GCCC-3′.

A seventeen-ribonucleotide sequence complementary to the 3′ terminus of Fragment 1 was added at the 5′ terminus to facilitate the assembly with Fragment 1.

The 3′-primer ACVR1-3 has the sequence of

(SEQ ID NO: 12)
5′-TATCACAGCTCTTGGGCTCCTCCAGGTGGAAGTTCTGGG-3′.

Similarly, a nineteen-ribonucleotide sequence complementary to the 5′ terminus of Fragment 3 was added at the 3′ terminus to facilitate the assembly with Fragment 3.

Synthesis of the cDNA of Fragment 3

The template plasmid for PCR amplification of Fragment 3 comprised the gene coding for the amino acid sequence (aa 216 to 447) of human IgG γ1 Fc (SEQ ID NO: 5).

The 5′-primer Fc-5 has the sequence of 5′-GAGCCCAAGAGCTGTGATA-3′ (SEQ ID NO: 13), which is complementary to the 3′ terminus sequence of the cDNA of Fragment 2.

The 3′-primer BGH-R has the sequence of

(SEQ ID NO: 14)
5′-AACTAGAAGGCACAGTCGAGGC-3′.

Assembly of the Fragments

Overlap extension PCR was conducted to link the cDNAs of Fragments 1 and 2. The two fragments were purified and used as templates, and the 5′-prime CMV-P for Fragment 1 and the 3′-primer ACVR1-3 for Fragment 2 were used for the PCR. The assembled PCR fragment was then linked to Fragment 3, using CMV-P and BGH-R as the primers.

Construction of the Recombinant Plasmid

The obtained PCR fragment was treated with restriction endonucleases NotI and XbaI. The ACVR1-Fc expression gene fragment was cloned into pcDNA3.1(Invitrogen), an expression vector effective in mammalian cells, using T4 DNA ligase. The anti-neomycin gene in pcDNA3.1 was replaced by the DHFR (dihydrofolate reductase) gene, and the modified vector is useful to screen for stably transformed mammalian cells. The recombinant plasmid was tranfected into DH5a competent cells. Clones positive for correct recombinant plasmids was identified by colony PCR. The recombinant plasmids were purified and digested, and correct sequence of the recominant gene was verified by sequencing.

### Example 2

### Construction of Expression Strain for the Fusion Protein

Host cell CHO DG44 (Item No. 12609-012, Invitrogen, USA) was cultivated through passages by following the manufacturer's instruction for CHO DG44. Non-transfected cells were cultivated in suspension in a CD DG44 culture medium (Invitrogen) supplemented with 8 mM L-glutamine and 5 μg/ml recombinant human insulin.

A CHO DG44 cell line capable of stable and high expression of the protein was constructed by stable transfection. Thus cloned CHO DG44 cells were cultivated in suspension in a culture medium free of serum and animal proteins.

The cell line stably expressing the fusion protein was constructed using the methods and steps as described below. Vector plasmids expressing the fusion protein were prepared using the Plasmid Maxi Preparation Kit from TianGen. The plasmids (100 μg) were treated with the restriction endonuclease Puvl to give linearized plasmids. The DG44 cells were cultivated through at least three passages before being transfected by the expression vector plasmid. The DG44 cells (1×10⁷ cells in total) were mixed with the digested plasmids in CD DG44 growth medium (0.8 ml), then transferred into a 0.4-cm pulsing cup (Bio-Rad). The cell/plasmid mixture was pulsed using a gene pulser (Bio-Rad, Gene Pulser Xcell). The transfected cells were cultivated in cell culture flask T-75 containing 20 ml growth medium. The flask T-75 with transfected cells was maintained in an incubator at 37° C., 8% CO₂ for 24 hours.

Transformants were selected by limiting dilution on a 96-well plate. The selection medium was OptiCHO comprising 8 mM L-glutamine, 5 μg/ml recombinant human insulin and 100 nM methotrexate (MTX). The cells were cultivated in an incubator at 37° C., 8% CO₂. Three weeks later, the culture fluid in each well positive for clone formation was analyzed by ELISA (alkaline phosphatase-conjugated goat-anti-human IgG Fc antibody, Jackson ImmuneResearch). The clones with high expression level of the protein were amplified and again, analyzed by ELISA. The amplification and analysis were repeated until stable high expression strains were obtained.

### Example 3

### Protein A Affinity Chromatography and HPLC-SEC of the Fusion Protein

The ACVR1-Fc fusion protein was purified from the supernate of the culture of stable expression cells using protein A affinity column according to the standard protocol (POROS, Mabcapture A). The purified protein was analyzed using reductive and non-reductive SDS-PAGE electrophoreses, as well as HPLC-SEC (high pressure liquid—molecule sieve).

### Example 4a

### ELISA Assay of the Fusion Protein's Binding to BMP-2 Protein In Vitro

In a 50 mM NaCO₃ solution, 3.5 μM recombinant human BMP-2 protein (Item No. 10426-HNAE, Sino Biological Inc. China) was dissolved. Aliquots (50 μl) of the BMP-2 protein solution were added onto a 96-well ELISA plate, and kept in refrigerator at 4° C. overnight. The next day, the ELISA plate was washed three times with TBST, before addition of 100 μl/well TBST with 3% BSA as blocking solution. The same number of blank wells, to which 100 μl of the blocking solution was added, were prepared to detect non-specific binding of ACVR1-Fc. The ELISA plate was placed in thermostat at 37° C. for 1 hr.

Dilutions of the fusion protein (3× serial dilution) were prepared in binding solution of TBST with 1% BSA. The blocking solution was decanted, and 50 μl/well of the 3× serial dilution of the fusion protein was added. The plate was placed in thermostat at 37° C. for 1 hr. After the fusion protein solution was decanted, the ELISA plate was washed three times with TBST, and then 50 μl/well of the secondary antibody (alkaline phosphatase-conjugated goat-anti-human IgG Fc antibody, Jackson ImmuneResearch) was added to react in thremostat at 37° C. for 1 hr. The developing antibody was removed, and to the ELISA plate, 200 μl/well TBST wash solution was added. The plate was then placed on a horizontal rotator at 100 rpm for 5 minutes, after which, the wash solution was decanted. The process was repeated five times. To the ELISA plate, 50 μl/well antibody developing solution (PNPP) was added. The ELISA plate was placed in thermostat at 37° C., and was read at 405 nm.

### Example 4b

### ELISA Assay of the Fusion Protein's Binding to other BMP/TGβ Family Members In Vitro

The recombinant human Activin A (Item no. 120-14E), BMP-5(Item no. 120-39), BMP-6 (Item no. 120-06) and BMP-7(Item no. 120-03) were all obtained from Peprotech (USA). These proteins are separately dissolved in a solution of NaCO₃ (20 mM, pH 9.6) to a concentration of 2 μg/ml. To a 96-well ELISA plate (Maxisorp, Nunc), 50 μl/well of the protein solution was added. The plate was then placed in refrigerator at 4° C. overnight. The next day, the ELISA plate was washed three times with PBST (PBS with 0.05% Tween-20), and then to the plate, 100 μl/well PBST (with 3% BSA) was added as blocking solution. The same number of blank wells, to which 100 μl of the blocking solution was added, were prepared to detect non-specific binding of ACVR1-Fc.

The ELISA plate was placed in thremostat at 37° C. for 2hr. Dilutions of the fusion protein (3× serial dilutions) were prepared in binding solution of PBST with 1% BSA. The blocking solution was decanted, and 50 μl/well of the 3× serial dilutions of the fusion protein was added. The plate was placed in thermostat at 37 ° C. for 2 hr. After the fusion protein solution was detanted, the ELISA plate was washed three times with PBST. Then, to the plate, 50 μl/well of the 3000× diluted secondary antibody (alkaline phosphatase-conjugated goat-anti-human IgG Fc antibody, Jackson ImmuneResearch) was added to react in thremostat at 37° C. for 2 hr. After the developing antibody was decanted, to the ELISA plate 200 μl/well PBST wash solution was added. The plate was placed on a horizontal rotator at 100 rpm for 5 minutes, after which, the wash solution was decanted. The process was repeated five times. To the ELISA plate, 50 μl/well antibody developing solution (PNPP) was added. The plate was placed in thermostat at 37° C., and was read on a microplate reader (iMax, Bio-rad) at 405 nm and 490 nm.

### Example 5

### Construction of the Recombinant Adenovirus Vector

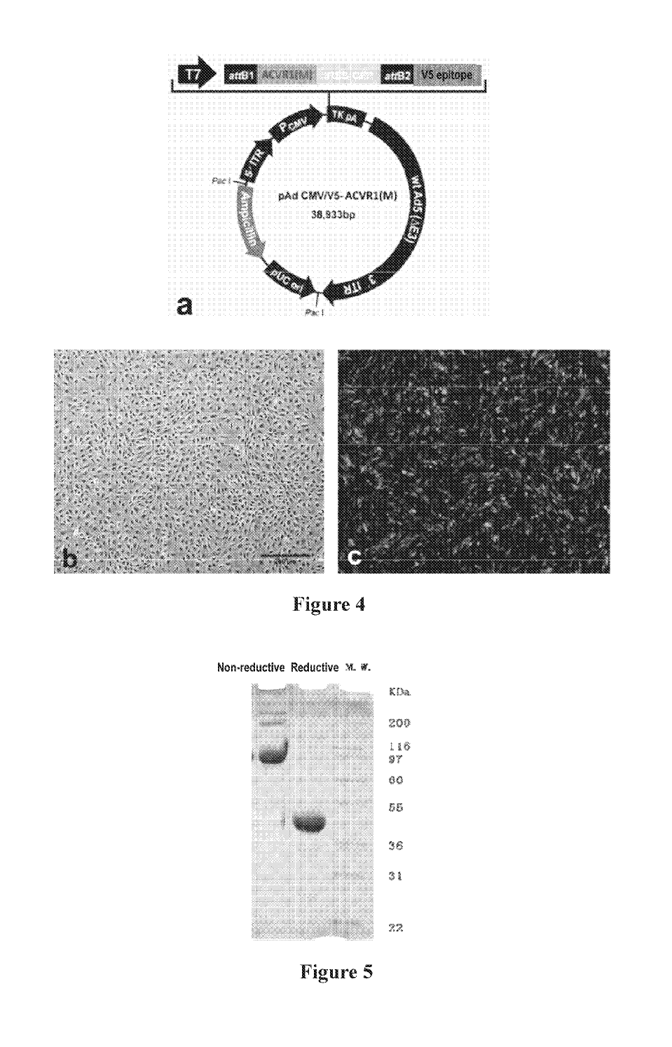

The ACVR1 gene was cut off from the vector Sport-ACVR1 (human) (Invitrogen) using restriction endonucleases Small and XhoI. G to A shift was made at position 617 in the obtained ACVR1 gene fragment using site-directed mutagenesis to obtain the ACVR1(M). The obtained ACVR1(M) fragment was cloned into plasmid pIRES2-EGFP (Invitrogen), which was then incorporated into pMD18-T simple vector (Takara) to obtain the recombinant plasmid. The obtained recombinant plasmid was packaged into pAd CMV/V5-DEST (Invitrogen) to construct the recombinant adenovirus of ACVR1(M)-IRES-GFP. The obtained virus vector was verified by DNA sequencing, and protein expression was verified by green fluorescence from GFP (FIG. 4). The obtained virus exhibited a titre of 1×10¹⁰ ifu/ml. FIG. 4a schematically illustrates the construction of the adenovirus plasmid expressing ACVR1 R206H.

### Example 6

### Induction of Osteogenic Differentiation and Chondrogenic Differentiation in HUVECs Infected with the Recombinant Virus

HUVECs (ATCC, CRL-1739) were maintained in EGM medium (Lonza, CC-3162) supplemented with 10% FBS (Gibco, 10099-141) and 1% penicillin-streptomycin (Gibco 15070-063). The cells were starved for 24 hr in Human Endothelial-Serum Free Medium (Gibco, 11111-044) supplemented with 2%FBS, 1% penicillin and streptomycin, as well as two growth factors, i.e., EGF (final concentration: 10 ng/ml) and bFGF (final concentration: 20 ng/ml), before being infected with the recombinant virus. Then, the ACVR1(M)-IRES-GFP adenovirus was added into the cells (MOI: 200, see FIG. 4c). After incubation for five days, the medium was replaced by osteogenic differentiation medium (Gibco StemPro osteogenic medium, A10072-01) or chondrogenic differentiation medium (Gibco StemPro osteogenic medium, A10071-01) for continued incubation to induce osteogenesis or chondrogenesis. The differentiation medium was changed to fresh every two days. Every experiment was conducted in triplicate, and repeated once.

### Example 7

### Evaluation of the Degree of Osteoblast Differentiation in the HUVEC Model—Alkaline Phosphatase Staining and Alizarin Red S Staining

In order to evaluate the degree of osteoblast differentiation in the HUVEC model obtained in Example 6, at day 7 and day 21 of the osteogenesis progress, the cells were stained with alkaline phosphatase (ALP) and Alizarin Red (Salizarin Red S). At day 7 of the incubation, the cells were washed three times with PBS, and then fixed with 4% formaldehyde. The cells were washed with PBS, before the substrate working solution was added, and the cells were then cultivated in dark for 30 minutes. At the end, the cells were washed with water, then examined and photographed under bright field microscope.

At day 21 of the osteogenic differentiation incubation, calcification of the cellular matrix was evaluated by Alizarin Red staining. As in the case of alkaline phosphatase staining, the cells were washed with PBS, fixed with 4% formaldehyde, washed with PBS again, and stained with 1% Alizarin Red S staining solution (pH 4.2) for 10 minutes. The cells were examined and photographed under bright field microscope.

### Example 8

### Evaluation of the HUVEC Model's Potential of Chondrogenic Differentiation—Alcian Blue Staining

At day 14 of the chondrogenic differentiation according to Example 6, the cells were harvested and washed three times with PBS, and fixed with 4% formaldehyde. The cells were washed three times again with PBS, and 0.3% Alcian Blue Stain 8GX (Sigma) was added for co-incubation. The cellular model's potential of chondrogenic differentiation was evaluated by detecting sulfated proteoglycan.

### Example 9

### Atomic Absorption Analysis of Calcium

Osteogenesis was induced on a 6-well plate by following the procedure of Example 6. At day 21 of the incubation, the cells were harvested, and calcium levels determined by atomic absorption were compared between cells.

The cells were washed three times with PBS (calcium- and magnesium-free), and then 1 ml lysis solution (0.1% Triton X-100, 10 mM Tris, pH 7.5) was added. The cells were decalcified using 11.6 N HCl at room temperature for 16 hr to release calcium as possible. The lysate was pipetted into a 1.5 ml Eppendoff, then centrifuged at 6000 rpm for 10 minutes. The supernate was collected and evaluated for calcium level by atomic emission spectrometer (Agilent, 7200).

### Example 10

### Levels of Osteogenic Differentiation Marker Proteins and Chondrogenic Differentiation Marker Proteins—Western Blotting

Western blotting was conducted to detect levels of osteogenic differentiation marker proteins and chondrogenic differentiation marker proteins, also the phosphorylation of BMP-Smad1/5/8. The cells were treated with RIPA lysis solution (containing the proteinase inhibitor PMSF), and supernate was collected via centrifugation at 13000 rpm for 5 minutes. Protein content in the supernate was determined by BCA assay. Lysates containing equivalent amounts of proteins were subjected to separation by SDS-PAGE electrophoresis. The electrophoresis was transferred onto the PVDF film, and the proteins were detected by Western blotting. The primary antibodies were specific for the marker proteins or phosphorylation of the proteins. The antibodies specific for the osteogenesis markers and the chondrogenesis markers were obtained from Abcam. The antibodies specific for the proteins in signaling pathway and for phosphorylation were obtained from Cell Signaling Technology. The secondary antibody was peroxidase-conjugated goat-anti-rabbit or goat-anti-mouse IgG (Jackson Immunoresearch Laboratories). Detection of the proteins was visualized by ECL Plus (Millipore).

Results obtained in Examples 1 to 10 are as discussed below:

Result 1: Identification of CHO DG44 as a Stable Expression Cell Line for ACVR1-Fc

By following the procedure of Example 2, a stable high expression strain for ACVR1-Fc was obtained, which provided an expression at the level of 600 mg/L.

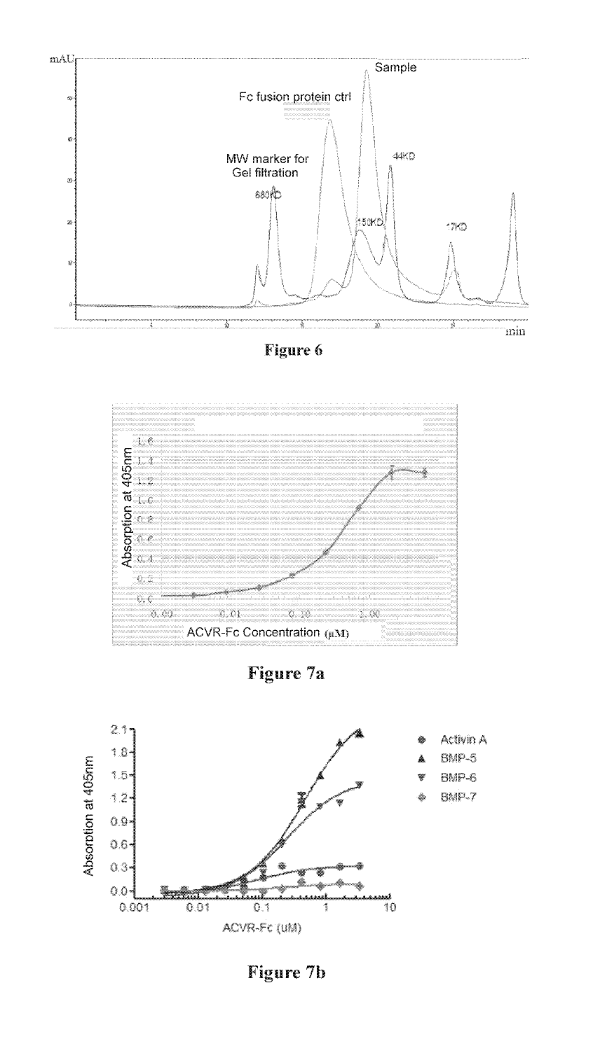

The fusion protein was purified through a protein A affinity column and then analyzed by reductive and non-reductive SDS-PAGE electrophoreses as described in Example 3. As shown in FIG. 5, the ACVR1-Fc protein is a dimer in its native state, with a molecule weight (MW) of 80 kDa, close to the theoretic value of 75 kDa.

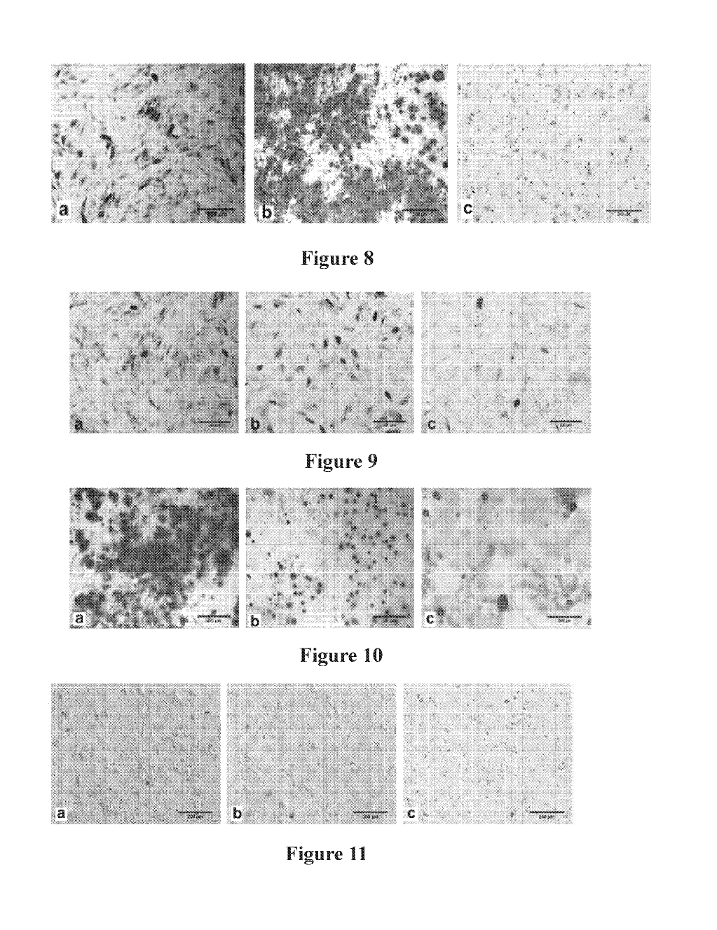

HPLC-SEC also indicated a MW around 80 kDa (FIG. 6). The results indicate that a stable expression strain was obtained, and the expressed ACVR1-Fc fusion protein is secretive and has a correct higher level structure.

Result 2a: ACVR1-Fc Binding to BMP-2 Protein In Vitro

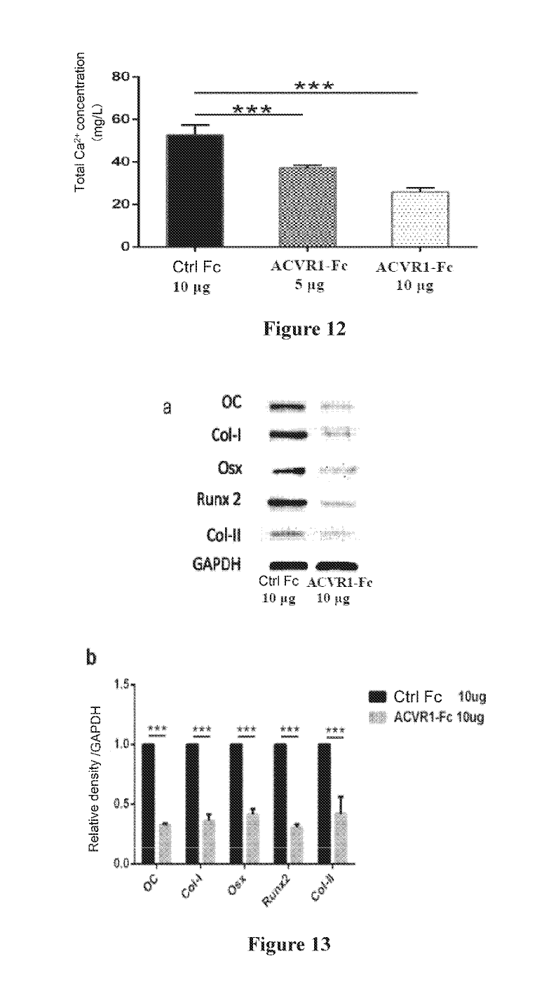

It has been shown that ACVR1 binds to bone morphogenetic protein-2(BMP-2). The ELISA in Example 4a indicates (FIG. 7a) that the ACVR1-Fc protein of the invention is capable of specifically binding to recombinant BMP-2 in vitro with an EC₅₀ of 0.42 μM.

Result 2b: ACVR1-Fc Binding to other BMP/TGFβ Family Members In Vitro

It has been shown that, several bone morphogenetic proteins, including BMP-2, BMP-5, BMP-6 and BMP-7, bind to ACVR1, resulting in activation of the BMP signaling pathway. The ELISA in Example 4b (FIG. 7b) indicates that the ACVR1-Fc protein of the invention is capable of binding to not only BMP-2 but also other BMP/TGFβ family members in vitro, though with different binding capacity and affinity. As shown, the ACVR1-Fc protein exhibited a much higher binding capacity to BMP-5 and BMP-6 than to BMP-7. This may be explained by the fact that the complex of ACVR1-Fc/BMP-5 or -6 exhibited a higher stability. However, the ACVR1-Fc protein exhibited the highest affinity to Activin A (EC₅₀ is 0.09 μM), and the affinity to the other proteins is not significantly different as compared to BMP-2, with the EC₅₀ values being 0.47 μM (BMP-5), 0.25 μM (BMP-6) and 0.21 μM (BMP-7), respectively.

The results indicate that the fusion protein of invention is capable of binding to different BMP/TGFβ family members, with differential capacity and affinity.

Up till now, there has only been reported that the protein complex ACVR-1/ACVR-2 binds to the members, like BMP-2, to effect the signaling. There has not been any report about whether ACVR-1 protein by itself is capable of binding to any BMP/TGFb proteins in vitro or in vivo. We find, for the first time, that ACVR-1 by it self is capable of binding to a BMP/TGFb proteins. Further, the in vitro biological activity data as provided suggests that the fusion protein of the invention is capable of inhibiting osteogenesis in cells carrying mutation(s) in ACVR-1. This also supports that the ACVR1-Fc protein of the invention binds to multiple BMP/TGFb proteins and has physiological effects.

Result 3: Construction of Osteogenic Differentiation Model in HUVEC

It has been shown that ACVR1 R206H is expressed in epithelia and induces the cells' transformation into stem-like cells, which are then capable of differentiating into osteoblasts and chondrocytes. In the present study, we reconstructed the HUVEC system to investigate the capability of the ACVR-1 fusion protein as a potential therapeutic to inhibit osteogenesis and chondrogenesis and to inhibit phosphorylation of the cells involved in cartilage formation and the BMP signaling pathway.

As shown in FIGS. 4b and 4c, the recombinant adenovirus constructed in Example 5 effectively infected the HUVECs to express ACVR1 R206H.

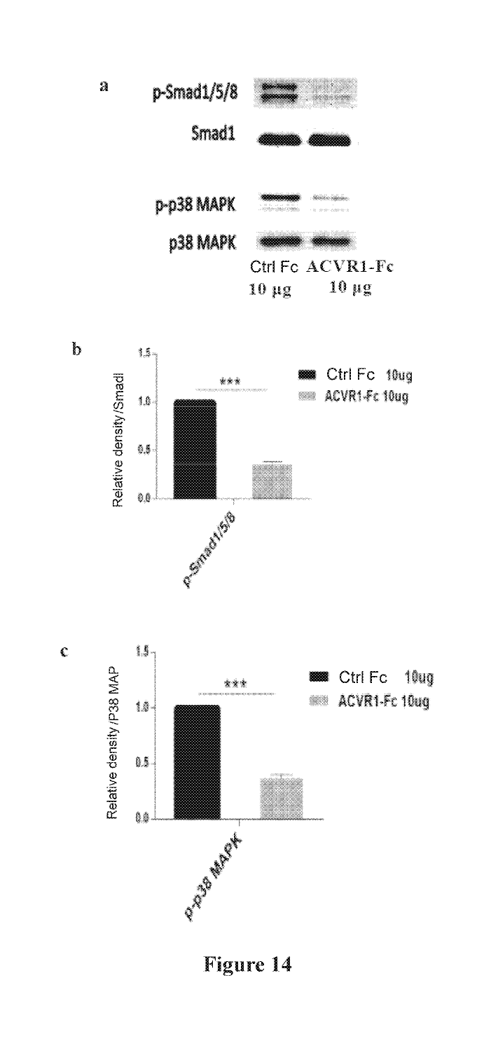

Staining of differentiated cells expressing ACVR1 R206H using alkaline phosphatase, Alizarin Red S and Alcian Blue are as shown in FIG. 8. As shown, the constructed ACVR1 R206H-expressing adenovirus induced HUVEC to differentiate into osteoblasts and chondrocytes.

Result 4: Study of ACVR1-Fc's Inhibition on Osteogenic Differentiation and Chondrogenic Differentiation

The alkaline phosphatase staining, the Alizarin Red S staining and the Alcian Blue staining (results shown in FIG. 9, FIG. 10 and FIG. 11) reflect the studies in Examples 7 and 8 on how the ACVR1-Fc protein impacts on osteogenesis or chondrogenic differentiation in HUVECs when being added during culture. The Fc control is a recombinant human IgG1 Fc protein (rhIgG1 Fc, Chimerigen Laboratories, Cat.# CHI-HF-210 IgG1).

As shown by FIG. 9 and FIG. 10, at day 7 of the differentiation, the cells exposed to the ACVR1-Fc protein exhibited a degree of differentiation lower than the cells exposed to the control protein (rhIgG1 Fc) (FIG. 9). The differentiation degree in HUVECs exhibited a negative correlation to the concentration of ACVR1-Fc in culture. The Alizarin Red staining at day 21 confirms the result (FIG. 10). It is thus concluded that addition of ACVR1-Fc inhibits osteogenic differentiation in HUVECs.

As seen in FIG. 11, at all the tested concentrations, the ACVR1-Fc fusion protein reduced chondrogenic differentiation compared to the control. At day 14, as shown by the Alcian Blue staining, the ACVR1-Fc protein also inhibited chondrogenic differentiation in HUVECs. It is thus concluded that the ACVR1-Fc protein inhibits chondrogenic differentiation in HUVECs.

Calcium ion level in intercellular matrix increases during osteoblast differentiation. At day 21, the concentration of calcium ion in intercellular matrix is determined via atomic absorption, as described in Example 9. As shown in FIG. 12, the calcium ion level in the calls treated with the ACVR1-Fc fusion protein is significantly lower that those treated with the Fc control. It is thus concluded that the ACVR1-Fc fusion protein inhibits osteogenic differentiation in HUVECs.

Result 5: Evaluation of Osteogenic Differentiation Marker Proteins

By following the procedure in Example 10, Western blotting was conducted to investigate effects the ACVR1-Fc protein has on expression of the markers of osteoblasts. The result is shown in FIG. 13. HUVECs were incubated in osteogenic differentiation media respectively containing the Fc protein as control and the ACVR1-Fc fusion protein for 9 days. The cells were then collected for Western blotting. As shown, the ACVR1-Fc protein reduced the expression of all the tested osteogenic differentiation markers. This is consistent with the fact that the ACVR1-Fc protein inhibited HUVEC osteogenic differentiation.

Result 6: Phosphorylation of the Proteins Involved in the Osteogenic Differentiation Signaling Pathway

The protein Smad-1/5/8 and the p38 MAPK signaling pathways are involved in osteogenic differentiation and chondrogenic differentiation. Accordingly, we evaluated whether ACVR1-Fc influences phosphorylation of protein Smad-1/5/8 and phosphorylation in the p38 MAPK pathway. Results are shown in FIG. 14.

As shown in FIG. 14, the ACVR1-Fc protein significantly inhibits phosphorylation of protein Smad-1/5/8 and the phosphorylation of p38 MAP kinase, and thus inhibits activation of p38 MAP kinase.

All documents identified in this disclosure are incorporated in their entirety herein by reference and as each is individually and specifically cited. In view of the description above, those of ordinary skill in the art will appreciate that various substitutions and/or other alterations may be made to the embodiments without departing from the spirit and scope of the invention as defined by the claims.

### APPEDIX 1. Brief Description of Sequence Identity

| SEQ ID NO: | Identity |
|---|---|
| 1 | coding sequence of Fragment 1 (signal peptide of CD33) |
| 2 | amino acid sequence of Fragment 1 |
| 3 | coding sequence of fragment 2 (the extracellular domain |
| | of ACVR1) |
| 4 | amino acid sequence of Fragment 2 |
| 5 | coding sequence of Fragment 3(human IgG γ1) |
| 6 | amino acid sequence of Fragment 3 |
| 7 | coding sequence of ACVR1-Fc fusion protein |
| 8 | amino acid sequence of CVR1-Fc fusion protein |
| 9 | 5′-primer for Fragment 1, CMV-P |
| 10 | 3′-primer for Fragment 1, SP-3 |
| 11 | 5′-primer for Fragment 2, ACVR1-5 |
| 12 | 3′-primer for Fragment 2, ACVR1-3 |
| 13 | 5′-primer for Fragment 3, Fc-5 |
| 14 | 3′-primer for Fragment 3, BGH-R |
| 15 | full-length amino acid sequence of ACVR1 |

### APPEDIX 2. References

1. Kaplan F S, Chakkalakal S A, Shore E M. Fibrodysplasia ossificans progressiva: mechanisms and models of skeletal metamorphosis. Dis Model Mech, 2012, 5: 756-762.
2. Pignolo R J, Shore E M, Kaplan F S. Fibrodysplasia ossificans progressiva: diagnosis, management, and therapeutic horizons. Pediatr Endocrinol Rev, 2013, 10 Suppl 2: 437-448.
3. Lounev V Y, Ramachandran R, Wosczyna M N. et al. Identification of Progenitor Cells That Contribute to Heterotopic Skeletogenesis. J Bone Joint Surg Am. 2009; 91:652-663.
4. Medici D, Shore E M, Lounev V Y, et al. Conversion of vascular endothelial cells into multipotent stem-like cells. Nat Med. 2010, 16(12):1400-1406.
5. Fiori J L, Billings P C, de la Peña L S, et al. Dysregulation of the BMP-p38 MAPK Signaling Pathway in Cells from Patients with Fibrodysplasia Ossificans Progressiva (FOP). J Bone Miner Res 2006, 21(6): 902-909.(MAPK inhibitor SB203580 was used in the present disclosure)
6. Billings P C, Fiori J L, Bentwood J L, et al. Dysregulated BMP Signaling and Enhanced Osteogenic Differentiation of Connective Tissue Progenitor Cells From Patients with Fibrodysplasia Ossificans Progressiva (FOP). J Bone Miner Res 2008, 23(3): 305-313.
7. Fukuda T, Scott G, Komatsu Y, et al. Generation of a Mouse with Conditionally Activated Signaling through the BMP Receptor, ALK2. Genesis, 2006, 44:159-167.
8. Yu P B, Deng D Y, Lai C S, et al. BMP type I receptor inhibition reduces heterotopic ossification. Nat Med. 2008, 14(12):1363-1369.
9. Glaser D L, Economides A N, Wang L, et al. In vivo somatic cell gene transfer of an engineered Noggin mutein prevents BMP4-induced heterotopic ossification. J Bone Joint Surg Am. 2003; 85:2332-42.
10. Kan L, Hu M, Gomes W A, et al. Transgenic mice overexpressing BMP4 develop a fibrodysplasia ossificans progressiva (FOP)-like phenotype. Am J Pathol. 2004; 165:1107-15.
11. Gomes W A, Mehler M F, Kessler J A. Transgenic overexpression of BMP4 increases astroglial and decreases oligodendroglial lineage commitment. Dev Biol 2003, 255:164-177.
12. Buczkowicz P, Hoeman C, Rakopoulos P, et al. Genomic analysis of diffuse intrinsic pontine gliomas identifies three molecular subgroups and recurrent activating ACVR1 mutations. Nat Genet. 2014, 46: 451-456.
13. Taylor K R, Mackay A, Truffaux N, et al. Recurrent activating ACVR1 mutations in diffuse intrinsic pontine glioma. Nat Genet. 2014, 46: 457-461.
14. Wu G, Diaz A K, Paugh B S, et al. The genomic landscape of diffuse intrinsic pontine glioma and pediatric non-brainstem high-grade glioma. Nat Genet. 2014, 46: 444-450.
15. Fontebasso A M, Papillon-Cavanagh S, Schwartzentruber J, et al. Recurrent somatic mutations in ACVR1 in pediatric midline high-grade astrocytoma. Nat Genet. 2014, 46: 462-466.
16. Tsai C L, Tsai C N, Lin C Y, et al. Secreted stress-induced phosphoprotein 1 activates the ALK2-SMAD signaling pathways and promotes cell proliferation of ovarian cancer cells. Cell Rep. 2012, 2: 283-293.
17. Cho H, Kim S, Shin H Y, et al. Expression of stress-induced phosphoprotein) (STIP1) is associated with tumor progression and poor prognosis in epithelial ovarian cancer. Genes Chromosomes Cancer. 2014, 53: 277-288.

1. A fusion protein including the following elements:
an ACVR1 element having the amino acid sequence ACVR1 or an active fragment thereof, preferably an ACVR1 extracellular segment sequence; and
an Fc element.

2. The fusion protein of claim 1, wherein the elements in the fusion protein are independently selected wherein:
the ACVR1 element is selected from:
a sequence having SEQ ID NO: 4;
a sequence as shown in SEQ ID NO: 4 and having one or more amino acid deletions, substitutions, or insertions and having the same biological activity as the sequence shown in SEQ ID NO: 4; and
the sequence shown in SEQ ID NO: 4 and having 90% or more homology to the sequence shown in SEQ ID NO: 4 and having the same biological activity as the sequence shown in SEQ ID NO: 4;
the Fc element is selected from the group consisting of an Fc fragment comprising human IgGγ1, IgGγ2, IgGγ3, IgGγ4
the signal peptide element is selected from the group consisting of:
a CD33 signal peptide;
a surface antigen protein signal peptide;
an antibody protein signal peptide; and
a secretion signal peptide molecule.

3. The fusion protein of claim 1, wherein the fusion protein is selected from the group consisting of:
a sequence having SEQ ID NO: 8;
a sequence having one or more amino acid deletions, substitutions, or insertions with the sequence shown in SEQ ID NO: 8, and having the same biological activity as the sequence shown in SEQ ID NO: 8; and
a sequence having at least 90% homology to the sequence shown in SEQ ID NO: 8 and having the same sequence as shown in SEQ ID NO: 8 sequences that have the same biological activity.

4. An isolated nucleic acid molecule which is the coding sequence of the fusion protein of claim 1 or is the complement of the coding sequence.

5. The nucleic acid molecule of claim 4, wherein the nucleic acid molecule further comprises:
the sequence set forth in SEQ ID NO: 3; and
the sequence set forth in SEQ ID NO: 5.

6. A vector wherein the vector contains the nucleic acid molecule according to claim 4.

7. A host cell wherein the host cell comprises the vector of claim 6.

8. The method of producing the fusion protein according to claim 1, the method including the steps of:
cultivating a host cell under conditions suitable for the expression of the fusion protein so as to express the fusion protein, the host cell including a vector, the vector containing a nucleic acid molecule that is one of a coding sequence of the fusion protein and a complement of the coding sequence of the fusion protein; and
isolating the fusion protein.

9. A medicament or a pharmaceutical composition for at least one of the group consisting of preventing a disease or condition associated with abnormal ACVR1 and treating a disease or condition associated with abnormal ACVR1, the medicament or a pharmaceutical comprising at least one of:
the fusion protein of claim 1;
a nucleic acid molecule that is one of a coding sequence of the fusion protein and a complement of the coding sequence of the fusion protein;
a vector containing the nucleic acid molecule; and
a host cell including the vector.

10. (canceled)

11. The fusion protein of claim 1, wherein the FC element is a human IgG Fc fragment.

12. The fusion protein of claim 1, further including a signal peptide element.

13. The fusion protein of claim 1, further including at least one linker peptide sequence located between the ACVR1 element and the Fc element.

14. The fusion protein of claim 2, wherein the Fc element is selected from the group consisting of:
an Fc fragment having the sequence of SEQ ID NO: 6;
an Fc fragment having one or more amino acid deletions, substitutions, or insertions into the sequence of SEQ ID NO: 6 and having the same biological activity as the sequence of SEQ ID NO: 6; and
a sequence having at least 90% homology to the sequence shown in SEQ ID NO: 6 and having the same biological activity as the sequence shown in SEQ ID NO: 6.

15. The fusion protein of claim 14, wherein the a secretion signal peptide molecule is a signal peptide having SEQ ID NO:2.

16. The medicament or pharmaceutical composition of claim 9, further comprising a pharmaceutically acceptable carrier.

17. The medicament or pharmaceutical composition of claim 9, wherein the disease or condition is selected from at least one of the group consisting of:
ossification or pleonosteosis associated diseases; and
at least one of the group consisting of cancers and over-activation associated with ACVR1.

18. The medicament or pharmaceutical composition of claim 17, wherein the at least one of the group consisting of cancers and over-activation associated with ACVR1 is at least one of:
high-grade gliomas, such as diffuse endogenous pontine gliomas; and
ovarian cancer.

19. (canceled)

20. A method for at least one of preventing a disease or condition associated with abnormal ACVR1 and for treating a disease or condition associated with abnormal ACVR1 in a subject, wherein the method comprises administering to the subject an effective amount of a medicament or pharmaceutical composition of claim 9.

21. The method of claim 20, wherein the disease or condition is selected from at least one of the group consisting of:
ossification or pleonosteosis associated diseases; and
at least one of the group consisting of cancers and over-activation associated with ACVR1, the at least one of the group consisting of cancers and over-activation associated with ACVR1 being at least one of:
high-grade gliomas, such as diffuse endogenous pontine gliomas; and
ovarian cancer.

22. The fusion protein of claim 12, further including:
at least one linker peptide sequence disposed between any two of the elements selected from the ACVR1 element;
the Fc element; and
the signal peptide element.

23. The nucleic acid molecule of claim 5, further comprising the sequence set forth in SEQ ID NO: 1.

24. The nucleic acid molecule of claim 23, wherein the nucleic acid molecule is selected from the group consisting of:
a sequence having SEQ ID NO: 7;
a nucleotide sequence having one or more nucleotide deletions, substitutions or insertions into the sequence set forth in SEQ ID NO: 7; and
a sequence having at least 90% homology to the sequence shown in SEQ ID NO: 7 and having the same biological activity as the sequence shown in SEQ ID NO: 7.


SEQUENCE LISTING (15 sequences)

SEQ ID NO: 1 - DNA, 48 bp, Homo sapiens
atgccgctgctgctactgctgcccctgctgtgggcaggggccctggct

SEQ ID NO: 2 - protein, 16 aa, Homo sapiens
MPLLLLLPLLWAGALA

SEQ ID NO: 3 - DNA, 309 bp, Homo sapiens
atggaggacgagaagcccaaggtgaaccccaagctgtacatgtgcgtgtgcgagggcctgtcctgcggcaatgaggaccactgcgagggccagcagtgcttctcctccctgtccatcaacgacggcttccacgtgtaccagaagggctgcttccaggtgtacgagcagggcaagatgacctgcaagacccctccctcccccggacaagctgtggagtgctgccagggcgactggtgcaacaggaacatcaccgcccagctgcccaccaagggcaagtccttccccggcacccagaacttccacctggag

SEQ ID NO: 4 - protein, 103 aa, Homo sapiens
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLE

SEQ ID NO: 5 - DNA, 699 bp, Homo sapiens
gagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagggaaataa

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1008 bp, Artificial Sequence
  coding sequence of ACVR1-Fc fusion protein
atggaggacgagaagcccaaggtgaaccccaagctgtacatgtgcgtgtgcgagggcctgtcctgcggcaatgaggaccactgcgagggccagcagtgcttctcctccctgtccatcaacgacggcttccacgtgtaccagaagggctgcttccaggtgtacgagcagggcaagatgacctgcaagacccctccctcccccggacaagctgtggagtgctgccagggcgactggtgcaacaggaacatcaccgcccagctgcccaccaagggcaagtccttccccggcacccagaacttccacctggaggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagggaaataa

SEQ ID NO: 8 - protein, 335 aa, Artificial Sequence
  amino acid sequence of CVR1-Fc fusion protein
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 21 bp, Artificial Sequence
  5′-primer for Fragment 1, CMV-P
cgcaaatgggcggtaggcgtg

SEQ ID NO: 10 - DNA, 16 bp, Artificial Sequence
  3′-primer for Fragment 1, SP-3
agccagggcccctgcc

SEQ ID NO: 11 - DNA, 35 bp, Artificial Sequence
  5′-primer for Fragment 2, ACVR1-5
gggcaggggccctggctatggaggacgagaagccc

SEQ ID NO: 12 - DNA, 39 bp, Artificial Sequence
  3′-primer for Fragment 2, ACVR1-3
tatcacagctcttgggctcctccaggtggaagttctggg

SEQ ID NO: 13 - DNA, 19 bp, Artificial Sequence
  5′-primer for Fragment 3, Fc-5
gagcccaagagctgtgata

SEQ ID NO: 14 - DNA, 22 bp, Artificial Sequence
  3′-primer for Fragment 3, BGH-R
aactagaaggcacagtcgaggc

SEQ ID NO: 15 - protein, 489 aa, Homo sapiens
MEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC